(19) United States
(12) Patent Application Publication
(10) Pub. No.: US 2017/0128018 A1
(43) Pub. Date: May 11, 2017

(54) OBJECT INFORMATION ACQUIRING APPARATUS

(71) Applicant: CANON KABUSHIKI KAISHA, Tokyo (JP)

(72) Inventor: Tatsuro Kato, Tokyo (JP)

(21) Appl. No.: 15/333,380

(22) Filed: Oct. 25, 2016

(30) Foreign Application Priority Data
Nov. 10, 2015 (JP) 2015-220510

(51) Int. Cl. A61B 5/00

(52) U.S. Cl. CPC A61B 5/708; A61B 5/0095; A61B 5/4312; A61B 5/444; A61B 2562/046; A61B 2576/02

(57) ABSTRACT

An object information acquiring apparatus of the present invention includes a supporter supporting a plurality of irradiators that each applies a light beam to an object and a probe that receives an acoustic wave from the object, an irradiation controller controlling the light beam from each of the plurality of irradiators, a movement controller moving position of the supporter, an acquirer acquiring information related to a light amount suppressing area, and an information processor generating characteristics information of the object, and the irradiation controller suppresses alight amount applied to the light amount suppressing area.

### BACKGROUND OF THE INVENTION

Field of the Invention

The present invention relates to an object information acquiring apparatus.

Description of the Related Art

A photoacoustic imaging method is proposed as a technique for imaging the inside of an object by using an acoustic wave. In the photoacoustic imaging method, a pulsed laser light beam is applied to the object, an acoustic wave generated from a living tissue having absorbed the energy of the light beams propagated and diffused in the object (this acoustic wave will hereinafter be also referred to as a photoacoustic wave) is detected, and information related to optical characteristic values inside the object is visualized. For example, when light with a wavelength that is adapted to be absorbed by hemoglobin is used as the pulsed laser light beam, it is possible to noninvasively obtain an image of a vessel in a living body.

In the case where the object is a breast, using a curved holder to hold the breast results in smaller pressure applied to the breast compared to using a flat holder, which also alleviates a burden on a subject. In the apparatus disclosed in Japanese Patent Application Laid-open No. 2012-179348, the breast is held with a cup-shaped holder and a photoacoustic wave from the breast is acquired by causing a light irradiator and a probe to integrally scan the holder.

Patent Literature 1: Japanese Patent Application Laid-open No. 2012-179348

### SUMMARY OF THE INVENTION

Here, since a nipple or a mole has a darker color than other portions of the subject, the light absorption amount of same is larger. Accordingly, the photoacoustic wave generated in accordance with light applied to the nipple or the mole is larger than those generated from the other portions. In addition, as a result of the nipple or the mole absorbing a large amount of light, the amount of light that reaches a deeper portion through the nipple or the mole is reduced. As a result, the photoacoustic wave from the deeper portion is reduced, and therefore accuracy degrades in the visualization of portions other than the nipple or the mole.

The present invention has been made in view of the above problem. An object of the present invention is to suppress the photoacoustic wave from the nipple or the mole and favorably acquire photoacoustic waves from other portions in photoacoustic imaging.

The present invention provides an object information acquiring apparatus comprising:

a light source;

a supporter that supports a plurality of irradiators that each applies a light beam from the light source to an object for irradiation and a probe that receives an acoustic wave that propagates from the object to which the light beam is applied;

an irradiation controller that controls the irradiation with the light beam from each of the plurality of irradiators;

a movement controller that moves a relative position of the supporter to the object;

an acquirer that acquires information related to a light amount suppressing area in the object; and

an information processor that generates characteristics information of the object based on the acoustic wave, wherein

the irradiation controller suppresses a light amount of the light beam applied to the light amount suppressing area by controlling each of the plurality of irradiators at each position of the supporter after the movement by means of the movement controller.

According to the present invention, it is possible to suppress the photoacoustic wave from the nipple or the mole and favorably acquire photoacoustic waves from other portions in photoacoustic imaging.

Further features of the present invention will become apparent from the following description of exemplary embodiments with reference to the attached drawings.

### BRIEF DESCRIPTION OF THE DRAWINGS

FIG. 1 is a view showing a configuration of a photoacoustic apparatus;

FIGS. 2A and 2B are views showing examples of the disposition of light irradiators and alight irradiation area;

FIGS. 3A and 3B are views for explaining movement of a high-resolution area;

FIG. 4 is a flowchart for explaining a setting method of an irradiation/non-irradiation area; and

FIGS. 5A and 5B are views for explaining a control method of irradiation/non-irradiation.

### DESCRIPTION OF THE EMBODIMENTS

Hereinbelow, preferred embodiments of the present invention will be described with reference to the drawings. Note that the dimensions, materials, shapes, and relative dispositions of components described below should be appropriately changed according to the configuration of an apparatus to which the invention is applied and various conditions. Therefore, the scope of the invention is not limited to the following description.

The present invention relates to a technique for detecting an acoustic wave that propagates from an object, and generating and acquiring characteristics information of the inside of the object. Therefore, the present invention is viewed as an object information acquiring apparatus or a control method thereof, or an object information acquiring method or a signal processing method. In addition, the present invention is also viewed as a program that causes an information processing apparatus including hardware resources such as a CPU and a memory to execute the methods, or a storage medium that stores the program.

The object information acquiring apparatus of the present invention includes an apparatus utilizing a photoacoustic effect that receives the acoustic wave generated in the object by irradiating the object with light (electromagnetic wave) and acquires the characteristics information of the object as image data. In this case, the characteristics information is information on characteristic values corresponding to a plurality of positions in the object that is generated by using a reception signal obtained by receiving a photoacoustic wave.

The characteristics information acquired by photoacoustic measurement is a value in which the absorption rate of light energy is reflected. The characteristics information includes, e.g., a generation source of the acoustic wave generated by light irradiation, an initial sound pressure in the object, a light energy absorption density and a light energy absorption coefficient derived from the initial sound pressure, and the concentration of a substance constituting a tissue. It is possible to calculate an oxygen saturation distribution by determining an oxygenated hemoglobin concentration and a reduced hemoglobin concentration as the substance concentration. In addition, a glucose concentration, a collagen concentration, a melanin concentration, and the volume fraction of fat or water are also determined.

A two-dimensional or three-dimensional characteristics information distribution is obtained based on the characteristics information at each position in the object. Distribution data can be generated as image data. The characteristics information may be determined not as numerical data but as distribution information at each position in the object. That is, distribution information such as an initial sound pressure distribution, an energy absorption density distribution, an absorption coefficient distribution, or the oxygen saturation distribution may be used as the characteristics information.

The acoustic wave in the present invention is typically an ultrasound wave, and includes an elastic wave called a sound wave or an acoustic wave. An electric signal obtained by converting the acoustic wave by a probe or the like is also referred to as an acoustic signal. Note that the description of the ultrasound wave or the acoustic wave in the present specification is not intended to limit the wavelength of the elastic wave. The acoustic wave generated by the photoacoustic effect is referred to as a photoacoustic wave or an optical ultrasound wave. An electric signal derived from the photoacoustic wave is also referred to as a photoacoustic signal.

In the following description and drawings, the same components are designated by the same reference numerals in principle, and the detailed description thereof will be omitted. In the following description, as an example of the object information acquiring apparatus, a photoacoustic apparatus that acquires the characteristics information of the inside of the object by using photoacoustic tomography and images the acquired characteristics information will be described. Note that, in the following description, a breast of a living body will be described as an representative example of the object, but the object is not limited thereto, and examples of the object include a hand and a leg.

### Embodiment 1

(Apparatus Configuration)

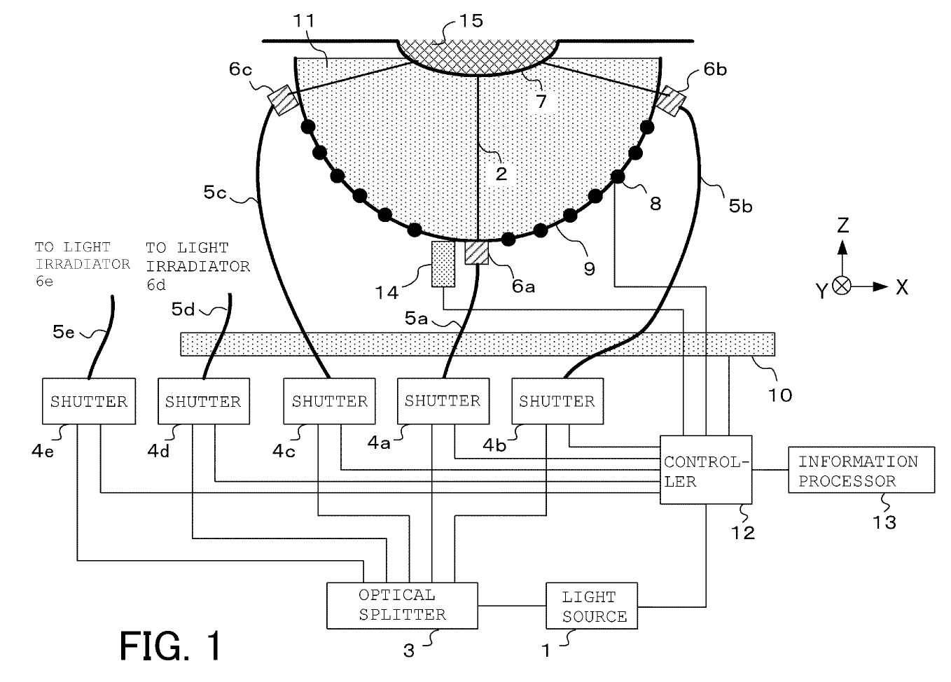

The photoacoustic apparatus shown in FIG. 1 includes a light source 1, an optical splitter 3, shutters 4a to 4e, optical transmitters 5a to 5e, light irradiators 6a to 6e, a holder 7, a probe 8, a probe supporter 9, a stage 10, an acoustic matching member 11, a controller 12, an information processor 13, and an optical imaging apparatus 14.

The light source 1 generates a laser light beam 2 (pulsed laser light beam) that is applied to an object 15 for irradiation. The light source 1 is a Ti:sapphire laser that outputs the laser light beam 2 having a center wavelength in the near-infrared region. Note that, as the light source 1, it is possible to use various lasers such as a solid state laser and a gas laser. In addition, instead of the laser, it is also possible to use a light-emitting diode and a flash lamp. The wavelength of irradiation light is selected in accordance with the type of a light absorber in the object 15 that serves as an imaging target. As the range of the selection of the wavelength, for example, the wavelength of 600 nm to 1100 nm is preferable. In addition, in order to efficiently generate the photoacoustic wave, a pulse width is preferably about 10 nanoseconds to 100 nanoseconds.

The laser light beam 2 emitted from the light source 1 is split into five laser light beams by the optical splitter 3. The optical splitter 3 is constituted by a beam splitter and a mirror. A plurality of the laser light beams 2 obtained by the splitting pass through the shutters 4a to 4e, and are transmitted to the light irradiators 6a to 6e by the optical transmitters 5a to 5e. The shutters 4a to 4e are configured to open and close with a signal from the controller 12, and are capable of switching between irradiation and non-irradiation of the laser light beam 2. As the optical transmitters 5a to 5e, a transmission by an optical fiber, a lens and a mirror, a space transmission that uses a diffusion plate, or a combination thereof is appropriate. At this point, the controller functions as an irradiation controller of the present invention.

Note that, instead of the shutters that switch between the irradiation/non-irradiation of light, a mechanism that continuously changes the transmission rate of light such as a diaphragm may also be provided. In addition, a variable beam splitter capable of continuously adjusting the transmittance of light may also be provided. Further, a plurality of optical attenuating mechanisms (filters or the like) having different transmission rates of light may be provided on each optical transmitter in advance, and a light amount may be changed stepwise by switching between the optical attenuating mechanisms. By providing at least one light amount switching mechanism described above, it becomes possible to perform adaptive light amount adjustment corresponding to the darkness of the color of the subject and the area thereof.

The light irradiators 6a to 6e are provided in the probe supporter 9 in order to guide the laser light beams 2 from the optical transmitters 5a to 5e to the object 15. As the material of each of the light irradiators 6a to 6e, glass or resin is preferable, but any material may be used as long as the material transmits the laser light beam 2.

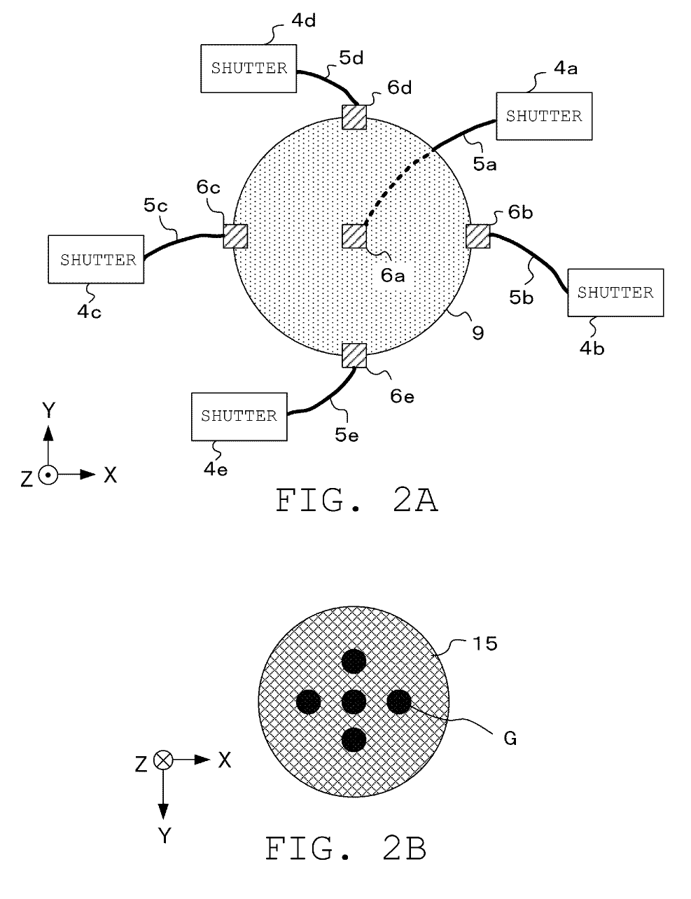

FIG. 2A is a top view of the probe supporter 9, and shows disposition positions of the light irradiators 6a to 6e. The light irradiator 6a is disposed at the bottom of the probe supporter 9. The light irradiators 6b to 6e are disposed at regular intervals near the edge of the probe supporter 9. The irradiation angles of the laser light beams 2 from the light irradiators 6a to 6e are adjusted such that irradiation areas do not overlap each other on the surface of the object 15. However, in the case where light is diffused or the case where the light source having a low degree of concentration such as a flash lamp is used, the peripheral edge portions of the irradiation areas sometimes overlap each other.

FIG. 2B is a bottom view of the object 15. Each of black areas G indicates the irradiation area on the object surface (holder surface) of each of the laser light beams 2 from the light irradiators 6a to 6e. As shown in the drawing, the irradiation areas do not overlap each other. In the present embodiment, although the laser light beams 2 are emitted from the five light irradiators 6a to 6e, the number of the light irradiators is not limited thereto, and it is only necessary to have two or more light irradiators. In accordance with the number of the light irradiators 6, the number of splits in the optical splitter 3 and the number of the optical transmitters 5 are adjusted. In addition, the disposition of the light irradiators 6 is not limited to the disposition of the present embodiment, and can be changed.

The laser light beams 2 emitted from the light irradiators 6a to 6e are applied to the object 15 held in the holder 7 for irradiation. By holding the object 15 with the holder 7, the surface shape of the object 15 is stabilized, and hence estimation of the light amount inside the object 15 is facilitated. Note that, even in the case where the holder 7 is not used, the processing of the present invention can be executed by acquiring the surface shape of the object by optical imaging or ultrasound wave transmission and reception.

As the holder 7, in order to cause the laser light beams 2 from the light irradiators 6a to 6e to reach the object 15, a member having high transmittance of the laser light beam 2 is used. Further, in order to cause the photoacoustic wave from the object 15 to pass through the holder 7, a material having an acoustic impedance close to that of the object 15 is preferable as the material of the holder 7. As the holder 7, it is possible to use, e.g., resin materials such as polymethyl pentene and polyethylene terephthalate, and elastic members such as latex and silicone. The holder preferably has a shape along the breast.

In order to efficiently receive the photoacoustic wave from the object 15 with the probe 8, it is preferable to bring the holder 7 into contact with the object 15 via the acoustic matching member such as liquid including water or gel.

The laser light beam 2 applied to the object 15 diffuses and propagates in the object 15. When part of energy of the light beam having diffused and propagated is absorbed by a light absorber such as blood, the photoacoustic wave is generated by the thermal expansion of the light absorber.

The photoacoustic wave generated in the object 15 is received by a plurality of the probes 8 disposed in the probe supporter 9. The probe 8 receives the photoacoustic wave generated on the surface of and in the object 15, and converts the photoacoustic wave to an electric signal. The probe 8 may be any probe such as the probe that uses a piezoelectric phenomenon or the probe that uses change of capacitance as long as the probe can receive the photoacoustic wave.

As the probe supporter 9, the one having high stiffness is preferable. As the material thereof, for example, metal is appropriate. In order to receive the photoacoustic wave generated in the object 15 at various angles, it is better to dispose a plurality of the probes 8 at various angles. Accordingly, in the present embodiment, the hemispherical probe supporter 9 is used. In addition, other than the hemispherical shape, it is also possible to use various shapes such as a spherical crown-like shape, a spherical zone-like shape, a bowl-like shape, part of an oval body, and a shape obtained by combining a plurality of planes or curves.

The acoustic matching member 11 is disposed so as to fill in the probe supporter 9, and connects the holder 7 and the probe 8 acoustically. The acoustic matching member 11 preferably transmits the light beams from the light irradiators 6a to 6e, and has the acoustic impedance close to those of the holder 7 and the probe 8. As the material of the acoustic matching member 11, water, gel, or oil is appropriate.

The electric signal of the photoacoustic wave outputted from the probe 8 is amplified by the controller 12, and the electric signal as an analog signal is converted to a digital signal. Note that, as functions of the controller 12 in the present specification, control of the light source and the light irradiator, signal processing such as amplification and conversion of the electric signal, stage position control, and nipple detection are described. However, the above control and processing may be performed by different processing apparatuses.

The information processor 13 acquires the initial sound pressure distribution as the characteristics information of the object 15 by using the digital signal obtained in the controller 12. In addition, the information processor 13 acquires a light absorption coefficient distribution of the object 15 by performing light distribution correction on the initial sound pressure distribution. At this point, it is possible to use existing image reconstruction methods such as back projection, delay and sum, and Fourier transformation. In the present invention, the presence or absence of the light irradiation from each irradiator (or the light amount) is set at each scanning position. The information processor 13 estimates a light amount distribution inside the object based on information related to the light irradiation, and uses the estimated light amount distribution in image reconstruction.

As the controller 12 or the information processor 13, it is possible to use a processor such as a CPU or a GPU, and an arithmetic circuit such as a field programmable gate array (FPGA) chip. Note that the controller 12 or the information processor 13 may also be constituted by a plurality of the processors or the arithmetic circuits instead of being constituted by one processor or one arithmetic circuit. These information processing apparatuses operate according to a program, and various functions are thereby implemented. Program modules for executing the individual steps of the program may be considered as separate configuration blocks. The apparatus preferably includes a memory that stores the electric signal, generated characteristics information and image data, and conditions related to the light irradiation. As the memory, it is possible to use a recording medium such as a ROM, a RAM, or a hard disk.

It is preferable to provide an inputting unit that receives an information input from a user (e.g., a doctor or an engineer) in the photoacoustic apparatus of the present invention. In the case where the controller 12 or the information processor 13 is constituted by the information processing apparatus such as a PC or a workstation, it is possible to use a user interface such as a mouse, a keyboard, or a touch panel as the inputting unit.

The reception surfaces of a plurality of the probes 8 are directed toward the center of the hemispherical probe supporter 9. In the case of this disposition, the center point of the hemisphere has the highest resolution, and the resolution is reduced with distance from the center point. An area in which the resolution is not less than a predetermined value (e.g., not less than the half of the highest resolution) is referred to as a high-resolution area.

The stage 10 moves the relative position of the high-resolution area to the object 15 by holding and moving the probe supporter 9. It is possible to move the high-resolution area by causing the stage 10 to scan the holder 7 in an X direction and in a Y direction to image the entire object 15 with high resolution. As the stage, it is possible to preferably use an XY stage that includes a guide, a ball screw, an alignment mechanism, and an actuator. Note that the movement of the high-resolution area may be one-dimensional movement or three-dimensional movement.

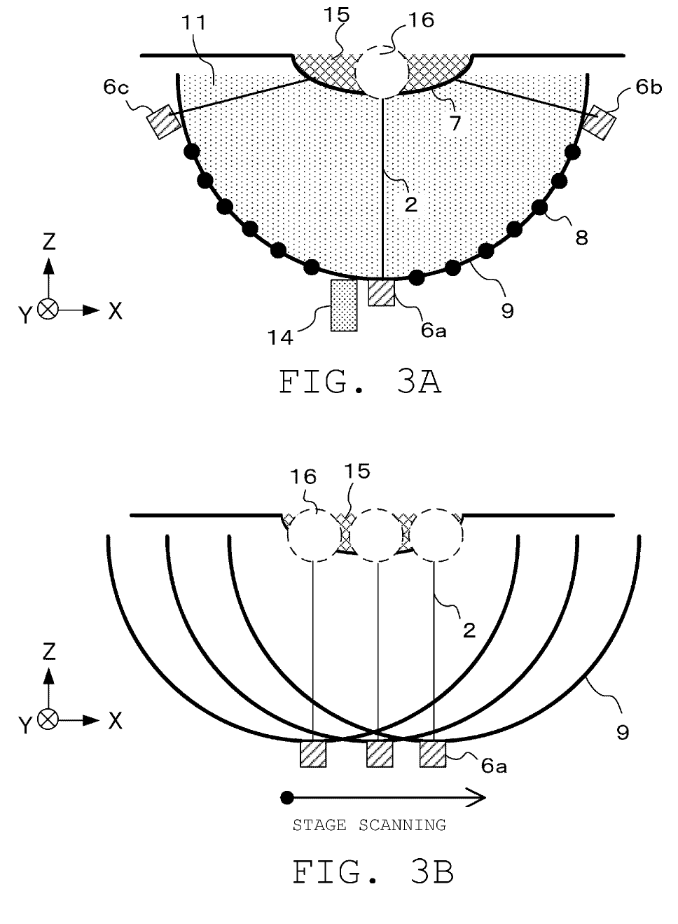

The movement coordinate of the stage 10 is controlled by the controller 12. At this point, the controller functions as a movement controller of the present invention. The apparatus emits the light beam at a plurality of movement positions and receives the photoacoustic wave. FIG. 3A shows an example of a high-resolution area 16. FIG. 3B shows a state in which the high-resolution area 16 moves with scanning of the stage 10.

At the bottom surface of the probe supporter 9, the optical imaging apparatus 14 for imaging the surface image of the object 15 is installed. Herein, as the optical imaging apparatus 14, a camera is used. When brightness for imaging the object 15 is insufficient, an illuminating unit may be provided and the object 15 may be illuminated by the illuminating unit. The image data acquired by the optical imaging apparatus 14 is inputted to the controller 12.

The controller 12 performs detection of a nipple or a mole based on the image data resulting from the imaging. At this point, the controller functions as an acquirer of the present invention. Note that a combination of the optical imaging apparatus and the controller may be considered as the acquirer. The detection target corresponds to a predetermined area of the present invention. The controller suppresses the light amount applied to the predetermined area such that the light amount is smaller than the light amount applied to an area other than the predetermined area. Consequently, the predetermined area is also referred to as a light amount suppressing area. The controller retains information related to the predetermined area (a position, a range, a shade, and the darkness of a color).

The predetermined area (the light amount suppressing area) may include an area having a color different from the color of a usual skin area such as a birthmark, a discolored portion, an areola, or a hair other than the nipple and the mole. In addition, the controller may acquire the predetermined area based on color references such a hue, a lightness, and a chroma instead of detecting the kind of a body tissue. For example, an area in which the lightness is not more than a predetermined threshold value is determined as the light amount suppressing target. As the color references, those preset and retained in the memory may be used, and values specified by the user via the inputting unit may also be used.

In addition, the user may specify the predetermined area by using the user interface. Examples of the specification method include a method in which the user specifies a range with the touch panel while watching an image obtained by optical imaging, and a method in which the user inputs numerical values of coordinates. The controller sets the area based on the inputted values.

(Setting Method of Irradiation/Non-Irradiation Area)

Hereinbelow, a setting method of the irradiation/non-irradiation area and control of irradiation/non-irradiation will be described.

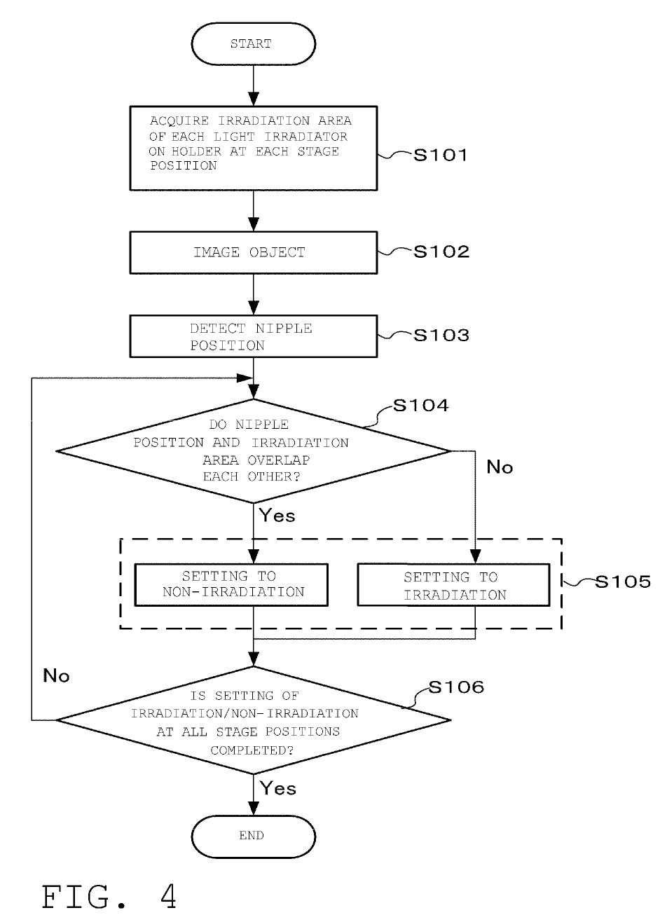

The setting method of irradiation/non-irradiation of the light irradiators 6a to 6e corresponding to the scanning position of the stage 10 will be described by using a flowchart in FIG. 4.

Prior to the measurement of the photoacoustic wave, an irradiation area on the holder 7 corresponding to the scanning position of the stage 10 is acquired for each of the light irradiators 6a to 6e (Step S101). In the acquisition of the irradiation area, it is possible to use a measuring unit such as a beam profiler. In addition, the irradiation area for each scanning position may be calculated based on the physical structure of the apparatus (a beam diameter, an irradiation direction, and the like) and the surface shape of the holder. In the case where the holder is used, this processing may be executed in advance before shipment or at the time of adjustment, and a table in which the scanning position is associated with the irradiation area may be retained in the memory. Even in the case where the holder is replaced in accordance with the size of the object, it is possible to create a table corresponding to each holder in advance.

Next, in Step S102, the surface of the object 15 is imaged by the optical imaging apparatus 14 (optical imaging unit), and the image obtained by the imaging is inputted to the controller 12.

In Step S103, the position and the range of the predetermined area are detected in the controller 12 based on the inputted obtained image. Herein, the predetermined area is assumed to denote the nipple and the mole. These areas have light absorption amounts larger than those of the other portions, and hence the amount of reflected light is small. Consequently, apart of the obtained image in which signal strength is small is determined, and the part thereof is detected as the nipple or the mole. Other than this method, any image analysis method may be used as long as the method is capable of detecting the predetermined area.

In Step S104, it is determined whether or not the irradiation area that overlaps the position of a nipple 17 is present when the irradiation areas of the light irradiators 6a to 6e and the position of the nipple 17 are projected on the holder 7. In the case where an overlap between the irradiation areas of the light irradiators 6a to 6e and the position of the nipple 17 is present (S104=Yes), any of the light irradiators 6a to 6e that causes the overlap is set to non-irradiation at the scanning position of the stage 10 that causes the overlap (Step S105).

On the other hand, the light irradiator that dose not cause the overlap is set so as to emit the light beam (S104=No). With regard to conditions of the irradiation/non-irradiation, a threshold value may be set such that the light irradiator is set to the non-irradiation in the case where a given percentage or more of the irradiation area overlaps the area of the nipple 17. In addition, the light irradiator may be set to the non-irradiation in the case where a distance between the centroid of the nipple 17 and the centroid of the irradiation area is shorter than a predetermined specified distance instead of using the state of the overlap. The method is not limited to the above methods as long as the method is capable of reducing the light amount applied to the light amount suppressing area.

Similarly, at each scanning position of the stage 10, information on the irradiation/non-irradiation of the laser light beams 2 from the light irradiators 6a to 6e is calculated. In Step S106, it is determined that the setting of the irradiation/non-irradiation at all of the scanning positions of the stage 10 is completed, and the processing is ended. The calculated information on the irradiation/non-irradiation is converted into a table, and the irradiation/non-irradiation of the laser light beams 2 from the light irradiators 6a to 6e is controlled at the time of the photoacoustic measurement based on the information in the table.

In addition, the information on the irradiation/non-irradiation converted into the table is used also for calculating the light absorption coefficient distribution of the object 15 in the information processor 13. The light absorption coefficient distribution is calculated by performing light distribution correction on the initial sound pressure distribution obtained by the image reconstruction. When a light distribution is calculated, the light amount applied to the object 15 is used. At this point, the light amount of the light irradiator set to the non-irradiation is assumed to be zero. Note that, when the information processor acquires the light distribution inside the object, simulation calculation such as a Monte Carlo method is performed based on the information on the irradiation/non-irradiation, the irradiation position, the surface shape of the object or the holder, and attenuation/dispersion characteristics of light inside the object. In the case where the light amount of each light irradiator is suppressed stepwise or continuously instead of the suppression based on the irradiation/non-irradiation, the light amount calculation corresponding to the change is performed.

(Control Method of Irradiation/Non-Irradiation of Light Irradiator)

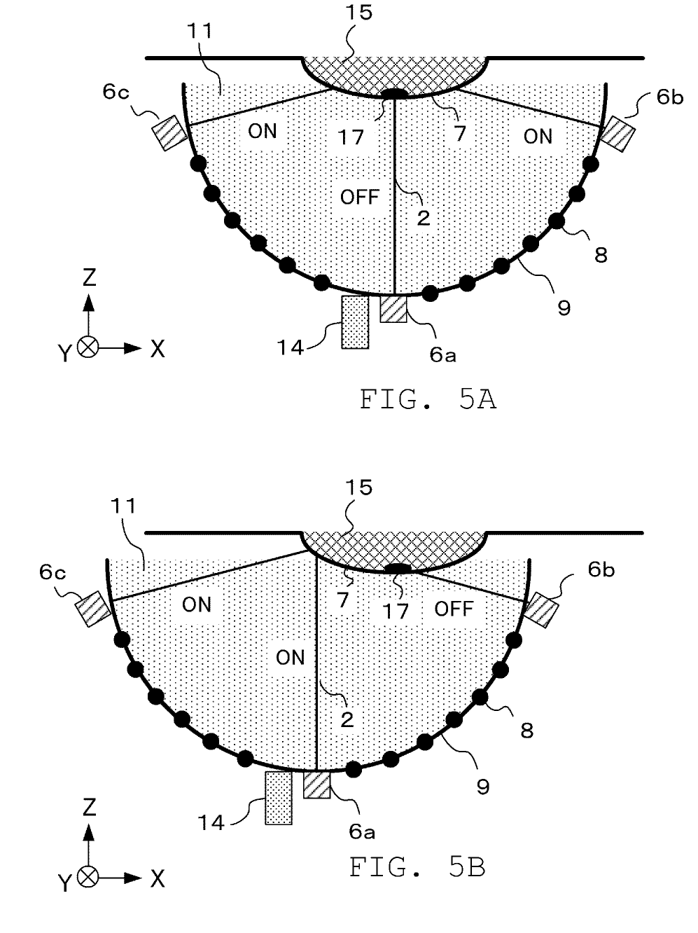

Subsequently, the control of the irradiation/non-irradiation of the light irradiators 6a to 6e will be described by using FIG. 5. In the present embodiment, the portion that is intended not to be irradiated (the light amount suppressing area) is the nipple 17.

FIG. 5A shows a state in which the laser light beam 2 emitted from the light irradiator 6a in the lower part of the drawing overlaps the position of the nipple 17. In this case, the laser light beam 2 from the light irradiator 6a is set to the non-irradiation. The controller 12 outputs a control signal to the shutter 4a such that the shutter 4a is closed. On the other hand, the laser light beams 2 from the other light irradiators 6b to 6e are set to the irradiation. That is, the controller 12 outputs the control signals to the shutters 4b to 4e such that the shutters 4b to 4e are opened. With this, the laser light beams 2 are emitted only from the light irradiators 6b to 6e.

FIG. 5B shows a state in which the stage 10 is moved in a −X direction from the state in FIG. 5A. In FIG. 5B, the laser light beam 2 emitted from the light irradiator 6b overlaps the position of the nipple 17. In this case, the controller 12 outputs the control signals to the shutters 4a to 4e such that the shutter 4b is closed and the other shutters 4a and 4c to 4e are opened. As a result, the light irradiator 6b is set to the non-irradiation, and the laser light beams 2 are emitted from the light irradiators 6a and 6c to 6e.

Thus, by controlling the irradiation/non-irradiation of the laser light beams 2 from the light irradiators 6a to 6e in accordance with the overlap information of the irradiation position and the nipple, the light irradiation to the nipple 17 is avoided. In addition, since a plurality of the light irradiators 6a to 6e are provided, even when the laser light beam 2 from a part of the light irradiators 6a to 6e is set to the non-irradiation, the laser light beams 2 from the other light irradiators 6a to 6e are emitted. With this, the laser light beam 2 is applied to a portion on an extension line that joins any of the light irradiators 6a to 6e that is set to the non-irradiation and the nipple 17 (a portion deeper than the nipple 17), and it is possible to acquire the photoacoustic wave from the portion deeper than the nipple 17.

Thus, according to the present embodiment, it becomes possible to acquire the photoacoustic wave from the other portions while suppressing the large photoacoustic wave from the nipple 17. As a result, it is possible to visualize the portion other than the nipple 17 with high accuracy. In addition, it is possible to achieve an effect of reducing photoacoustic measurement time by irradiating the object 15 by using a plurality of the light irradiators 6a to 6e.

(Modification)

In the present embodiment, in the case where the laser light beam 2 from a given light irradiator overlaps the nipple 17, the light irradiator is set to the non-irradiation, but the light irradiator may also be adjusted such that the irradiation light amount is reduced. In addition, in the present embodiment, the position of the nipple or mole is detected based on the image obtained by the optical imaging, but the invention is not limited thereto. For example, a mark for positioning is provided on the holder 7, and the position of the breast is adjusted such that the position of the nipple matches the mark. In this case, the optical imaging apparatus 14 and the function of detecting the position of the nipple based on the obtained image become unnecessary.

The color of the skin or the like varies from person to person. For example, there is a possibility that some people have the same darkness and brightness of the color of the skin as those of the nipple or the mole. In this case, the light absorption amount at the nipple or the mole is equal to those of the other portions. Consequently, the function of avoiding the light irradiation to the portion having the large light absorption amount becomes unnecessary. In preparation for such a case, it is preferable to provide a unit for turning off the above-described light amount suppressing function of the present invention (function switching unit).

For example, when the photoacoustic measurement is started, a measurement operator determines use/non-use of the present function, and performs setting from the user interface. In the case where the non-use is set, a series of functions including the imaging of the object 15 in the optical imaging apparatus 14, the detection of the area of the nipple or the mole, and the setting of the irradiation/non-irradiation of the light irradiators 6a to 6e are turned off. Alternatively, the function switching unit may automatically turn off the functions in accordance with the result of the optical imaging. The function switching unit can be constituted as one module of the information processing apparatus or a processing circuit separate from the information processing apparatus.

When the irradiation is stopped or the light amount is suppressed in any of the light irradiators 6a to 6e, the total light amount applied to the object is reduced. As a result, there is a possibility that the SN ratio of generated image data is reduced. To cope with this, the reduced light amount may be compensated by an output from the other light irradiators that execute the irradiation. The controller 12 selects the light irradiator that is subjected to increase control of the light amount based on the disposition place of the light irradiator. At this point, the controller 12 performs control such that the laser output from each light irradiator does not exceed the maximum permissible exposure.

In addition, as another method, the total light amount applied to the object may be made constant by fixing the number of the light irradiators that perform the irradiation concurrently. In the case where the light amount suppressing area is present in the object, at each scanning position, the light irradiation of at least one of a plurality of the light irradiators is not performed. In the case where the light amount suppressing area is not present in the object or the case where the function of suppressing the light amount is disabled, the light irradiation may be performed from all of the light irradiators. In either case, the controller 12 performs the control such that the laser output from each light irradiator does not exceed the maximum permissible exposure.

The present invention can be implemented by processing in which a program for implementing one or more functions of the above embodiments is supplied to a system or an apparatus via a network or a storage medium, and one or more processors in a computer of the system or the apparatus read and execute the program. In addition, the present invention can also be implemented by a circuit (e.g., ASIC) that implements one or more functions.

### OTHER EMBODIMENTS

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-220510, filed on Nov. 10, 2015, which is hereby incorporated by reference herein in its entirety.

What is claimed is:

1. An object information acquiring apparatus comprising:
a light source;
a supporter that supports a plurality of irradiators that each applies a light beam from the light source to an object for irradiation and a probe that receives an acoustic wave that propagates from the object to which the light beam is applied;
an irradiation controller that controls the irradiation with the light beam from each of the plurality of irradiators;
a movement controller that moves a relative position of the supporter to the object;
an acquirer that acquires information related to a light amount suppressing area in the object; and
an information processor that generates characteristics information of the object based on the acoustic wave, wherein
the irradiation controller suppresses a light amount of the light beam applied to the light amount suppressing area by controlling each of the plurality of irradiators at each position of the supporter after the movement by means of the movement controller.

2. The object information acquiring apparatus according to claim 1, wherein
the acquirer acquires the information related to the light amount suppressing area based on a light absorption amount on a surface of the object.

3. The object information acquiring apparatus according to claim 1, wherein
the object is a breast, and
the acquirer acquires at least information related to an area of a nipple of the breast.

4. The object information acquiring apparatus according to claim 1, wherein
the acquirer acquires at least information related to an area of a mole of the object.

5. The object information acquiring apparatus according to claim 1, further comprising:
an optical imaging unit that images the object, wherein
the acquirer acquires the information related to the light amount suppressing area by using an image of the object obtained by the imaging.

6. The object information acquiring apparatus according to claim 1, further comprising:
an inputting unit that receives an input from a user, wherein
the acquirer acquires the information related to the light amount suppressing area based on a specification from the user that uses the inputting unit.

7. The object information acquiring apparatus according to claim 1, wherein
the irradiation controller suppresses the light amount in a case where the light beam emitted from the irradiator overlaps the light amount suppressing area.

8. The object information acquiring apparatus according to claim 7, wherein
the irradiation controller suppresses the light amount by setting the light beam to non-irradiation by using a shutter.

9. The object information acquiring apparatus according to claim 7, wherein
the irradiation controller suppresses the light amount continuously or stepwise.

10. The object information acquiring apparatus according to claim 1, further comprising:
a holder that holds the object.

11. The object information acquiring apparatus according to claim 10, wherein
the irradiation controller controls the irradiation with the light beam based on an irradiation area of the light beam emitted from the irradiator on a surface of the holder.

12. The object information acquiring apparatus according to claim 11, wherein
the irradiation controller suppresses the light amount in a case where a distance between a centroid of the irradiation area and a centroid of the light amount suppressing area is shorter than a predetermined specified distance.

13. The object information acquiring apparatus according to claim 1, further comprising:
a function switching unit that turns off a function of the irradiation controller to suppress the light amount.

14. The object information acquiring apparatus according to claim 13, wherein
the acquirer turns off the function of the irradiation controller to suppress the light amount, in accordance with a specification from a user that uses an inputting unit that receives an input from the user.

15. The object information acquiring apparatus according to claim 13, wherein
the acquirer turns off the function of the irradiation controller to suppress the light amount, based on an image of the object acquired by an optical imaging unit that images the object.

16. The object information acquiring apparatus according to claim 1, wherein
the information processor
acquires an initial sound pressure distribution inside the object by image reconstruction that uses an electric signal obtained by converting the acoustic wave by the probe,
estimates, by using the light amount of the light beam emitted from each of the plurality of irradiators at each position of the supporter after the movement by means of the movement controller that is controlled based on the information related to the light amount suppressing area by the irradiation controller, a light distribution inside the object at the each position, and
acquires an absorption coefficient distribution inside the object by using the initial sound pressure distribution and the light distribution.

17. The object information acquiring apparatus according to claim 1, wherein
the irradiation controller performs, in a case where the plurality of irradiators include the irradiator of which the irradiation with the light beam is suppressed, compensation based on an output from another irradiator.
